(19) United States
(12) Patent Application Publication
(10) Pub. No.: US 2026/0108669 A1
(43) Pub. Date: Apr. 23, 2026

(54) AN ENEMA NOZZLE AND AN ENEMA DEVICE COMPRISING SAID ENEMA NOZZLE

(71) Applicant: Qufora A/S, Allerød (DK)

(72) Inventors: Henrik Bork Bjerregaard, Lynge (DK); Kristian Bjerg, Kokkedal (DK)

(21) Appl. No.: 19/162,007

(22) PCT Filed: Mar. 7, 2024
(86) PCT No.: PCT/EP2024/055955

§ 371 Date: Sep. 3, 2025

(30) Foreign Application Priority Data
Mar. 8, 2023 (DK) PA202370123

(51) Int. Cl. A61M 3/02

(52) U.S. Cl. CPC A61M 3/0279; A61M 2207/00

(57) ABSTRACT

An enema nozzle and an enema device comprising said enema nozzle The present invention relations to an enema nozzle (1; 31; 34; 35) arranged for delivering an enema to a user, said enema nozzle comprises a ball-shaped insertion tip (3) and an elongated insertion body (2) having the shape of a truncated cone, and wherein the cross-sectional diameter (A, B) of the insertion body (2) is smaller than the cross-sectional diameter (C) of the ball-shaped insertion tip (3). When the enema nozzle (1; 31; 34; 35) is inserted into a user's rectum, the user's anal sphincter will hold itself in the substantially contracted condition thereby X, Z both keeping the enema nozzle (1; 31; 34; 35) in place, and prevent any leakage of fluids.

### CROSS REFERENCE TO RELATED APPLICATIONS

This application is a U.S. National Phase Application of PCT International Application No. PCT/EP2024/055955, filed on Mar. 7, 2024, which is an International Application of and claims the benefit of priority to Danish Patent Application NO. PA 2023 70123, filed on Mar. 8, 2023. The entire contents of these patent applications are herein incorporated by reference.

The present invention relates to an enema nozzle for delivering an enema, and an enema device comprising said enema nozzle.

Administrating an enema is a common medical procedure whereby fluid is injected into the rectum and lower intestine of a patient in order to induce bowel movement. The need for such a procedure typically arises in patients suffering from certain physical ailments in which voluntary bowel control is impaired or when the bowel needs to be cleaned before e.g. a coloscopy or a surgical operation.

Enemas are often administered to a patient at home when the need for medical assistance does not necessitate a doctor or another health care assistant. In this respect it is often difficult for the patient to administer the enema liquid to himself or herself since the conventional enema devices often causes discomfort and irritation when being inserted. Moreover, it is difficult for the patient to administer the liquid while steadily holding the enema nozzle in place during the procedure. Often another individual assists the patient but assistance may not always be available, if for instance, the patient lives alone.

In order to overcome these problems a number of enema devices, comprising different retention means for retaining the nozzle in the rectum/colon during administration of the enema, have been developed.

Such a device is e.g. disclosed in US Pat. No. 5,074,842 which describes an irrigation device having a fixation balloon that is inflated using air, and wherein the air is delivered through a syringe valve communicating with an air passage. Similar systems are known from EP1531885, which describes an irrigation device comprising a liquid reservoir and an air-inflated balloon for fixation of a tubular part inserted in the rectum, and WO2011023196, which describes a system which uses liquid to inflate the balloon/retention member.

Since air or liquid is used to inflate the retention member/balloon in these known devices, these know enema systems are relatively complex constructions which may be difficult and time consuming to use. Not only must the device be arranged for delivering air/liquid to the balloon for retaining the enema nozzle in the body opening to be irrigated, but said air/liquid must also be removed from said balloon when the enema nozzle has to be removed from the body opening. The air or liquid may either be vented to the surroundings or delivered to the rectum and/or colon before the nozzle is removed from the rectum, and the user needs to be sure that the balloon in completely deflated in order to prevent damage to e.g. the rectal wall.

Furthermore, use of an air-filled balloon has the problems that the balloon will react to the body movements, e.g. peristaltic reflexes, whereby the nozzle inconveniently may be displaced causing leakage, and the air from the balloon cannot be delivered to the colon as this will result in an additional distention or dilation of the colon resulting in pain and discomfort for the patient.

A further problem with the use of inflated balloons is that if the balloon is overfilled the balloon may rupture causing both damage to the fragile wall of the colon/rectum and leakage of the liquid filled content of the colon/rectum. Furthermore, if the balloon is filled too little it cannot safely keep the enema nozzle in place during use. This is not only unwelcoming for the patient but also very demeaning, as the surroundings inevitability will be contaminated with irrigation liquid and bodily excretions if the enema nozzle unintentionally u falls out.

It is therefore a first aspect of the present invention to provide an enema nozzle which can be retained in the colon/rectum without use of an expandable retention means e.g. an inflatable balloon.

It is a second aspect of the present invention to provide an enema nozzle arranged for being connected to an enema device, which nozzle that can be inserted into an opening to be irrigated, without causing discomfort, trauma and/or injuries.

It is a third aspect of the present invention to provide an enema nozzle that is inexpensive to manufacture and is simple and reliable to use.

It is a fourth aspect of the present invention to provide an enema nozzle that is relatively small and easy to operate.

These and further aspects are achieved according to the present invention by providing an enema nozzle arranged for delivering an enema to a user, said enema nozzle comprises a ball-shaped insertion tip and an elongated insertion body having the shape of a truncated cone, and wherein the cross-sectional diameter of the insertion body is smaller than the cross-sectional diameter of the ball-shaped insertion tip.

Within the context of the present invention the term “cross-sectional diameter” of the elongated insertion body and the ball-shaped insertion tip means a diameter taken in a plane perpendicular to the longitudinal axis of the elongated insertion tip. Furthermore, the term “ball-shaped” means that the insertion tip has the shape of a sphere or a substantial sphere e.g. a spheroid (oblate or prolate) having a minor or major axis that differ less than 5% from each other. The term “insertion tip” refers to the distal section/end of the enema nozzle, i.e. the part which is inserted first in the rectum/colon, and the term “insertion body” refers to the section of the enema nozzle (below the ball-shaped insertion tip) that is intended for being placed at and inside the anal opening.

The inventors of the present invention has surprisingly found that such an enema nozzle will activate the user's anal sphincters, and ensure that said muscles are contracted and maintained in a substantially contracted condition a long as said enema nozzle is placed in the user's anus/rectum.

Thus, the enema nozzle according to the invention take advantage of the function of the anal sphincters which effectively will keep fluids, e.g. bodily excretions, liquid and gas from escaping unexpectedly from the colon.

The anal sphincters are a short tube of muscles surrounding the inferior portion of the anal canal and control the release of stool, thereby maintaining fecal continence. The anal sphincters comprise two muscles: the internal anal sphincter and the external anal sphincter. The external anal sphincter is largely under voluntary control by the somatic nervous system which allows it to stay in a contracted state, whereas the control of the internal sphincter is involuntary as it is innervated by the autonomic nervous system. The internal anal sphincter is tonically contracted most of the time to prevent leakage of fluid or gas, but is relaxed upon distention of the rectal ampulla, e.g. when feces are build up in said cavity.

The inventors of the present invention have surprisingly found that when the enema nozzle according to the invention is inserted into a user's anus, said enema nozzle does not affect the function of the anal sphincters and the user's anal sphincter will accordingly remain contracted and hold itself in the substantially contracted condition thereby not only keeping the enema nozzle in place, but also ensure that the irrigation liquid and bodily excretions are maintained in the rectum and colon during the enema administration procedure.

It has been found that the conventional enema nozzles are incapable of being retained in rectum/colon in a similar manner as the enema nozzle according to the invention. This is likely due to the form (such as a trumpet head) and/or relatively large cross-sectional diameter (more than 1 cm at least on one or more sections of the part which is inserted into the rectum/colon) of the insertion body/catheter of these conventional enema nozzles, as such constructions/dimensions will stretch the anal sphincter muscles whereby the sphincters cannot contract sufficiently to maintain the enema nozzle in place and/or the fluids in the rectum.

Thus, the conventional enema nozzles cannot be held in place (retained) simply by means of the function of the anal sphincters. As a result, the conventional enema nozzles require the aid of more or more retention member(s) that can expand when placed in the rectum. Furthermore, leakage of the enema fluid commonly occurs using these conventional enema nozzles, since the anal sphincters cannot provide a tight fit around the conventional enema nozzles, and this may led to insufficient colonic filling. Accordingly, imperfect procedural results may be obtained, or an insufficient dosage of enema (or medicine if this is administered) may be received by the patient. The enema nozzle according to the invention effectively prevents said problems as said enema nozzle simply uses the users own anal sphincters both to keep the enema nozzle safely in place and to prevent any leakage.

The ball-shaped insertion tip is located at the insertion end of the insertion body, and said tip will provide a smoother insertion, which effectively will alleviate the physical discomfort and pain that a patient may expire during insertion of a conventional enema nozzle.

Furthermore, since the insertion tip is ball-shaped, and preferably contains no sharp edges, any injuries to the rectum and colon walls are minimized. It is in this respect known that repetitive tissue trauma, which are not allowed time to heal, can result in more serious conditions. This is of particular concern with pediatric patients, who commonly move about during the enema procedure, as it is important to avoid having the enema nozzle rupture a fragile rectum wall. The enema nozzle according to the present invention effectively eliminates these problems as the ball-shaped insertion tip expediently reduces this risk. Repetitive enema administrations can thereby be performed without inflicting trauma to surrounding tissue using the enema nozzle according to the invention.

The inventors of the present invention have also found that the insertion of the enema nozzle into the anus/rectum of the patient is even easer than with the known enema nozzles in the art. The use of a ball-shaped insertion tip having a larger cross-sectional diameter than the elongated truncated insertion body ensures that during insertion the squeezing action of the anal sphincters against the insertion body pushes (sucks) the enema nozzle into the rectum, thereby ensuring that the enema nozzle according to the invention will be placed in the correct position in the rectum/colon.

It is preferred that the cross-sectional diameter of the insertion body is smaller than the cross-sectional diameter of the ball-shaped insertion tip in the entire length of the insertion body. Thus, the ball-shaped insertion tip is larger in cross-sectional diameter than the insertion body irrespectively of where the cross-sectional diameter is measured along the elongated insertion tip. As discussed above, it is believed that this specific constructions aids in obtaining the squeezing action of the anal sphincter against the insertion body which ensures that the enema nozzle according to the invention is “sucked” into the rectum, thereby ensuring that the enema nozzle according to the invention will be placed in the correct place in the rectum.

In some embodiments the enema nozzle may comprise a flared stop plate arranged for preventing the enema nozzle from being pushed and/or sucked to far into the rectum. Said flared stop plate is preferably placed adjacent to and just below the bottom circular base of the elongated insertion body.

Since the elongated insertion body is formed as a truncated cone it will have a top circular base and a bottom circular base, and the top circular base will have a smaller radius/diameter than the bottom circular base.

In a preferred embodiment the elongated insertion body is connected to the ball-shaped insertion tip in such a way that the top circular base of the insertion body i.e. the part of the insertion body with the smallest diameter, is connected/attached to the ball-shaped insertion tip, and the side face of the insertion body extended gradually outwards starting from the top circular base towards the bottom circular base with a larger diameter.

In a preferred embodiment the ball-shaped insertion tip has a cross-sectional diameter which is about 1.2 to 7 times larger than the cross-sectional diameter of the top circular base of the insertion body, preferable between 2 and 5 times, and even more preferred about 2.6 times the cross-sectional diameter of the top circular base of the insertion body, as it has been shown that such ratios are especially advantageously for ensuring that the enema nozzle according to the invention is correctly and sealing retained in the rectum during administration of an enema.

The bottom circular base may preferably have a cross-sectional diameter which is about 1.1 to 3.5 times larger than the cross-sectional diameter of the top circular base, preferable about 1.2 to 2 times, even more preferred about 1.6 times larger than the cross-sectional diameter of the top circular base. The cross-sectional diameters of the top and bottom circular bases are selected in order to ensure that the ball-shaped insertion tip will have a cross-sectional diameter which is larger than cross-sectional diameter of the elongated insertion body, preferably along (in) the insertion body's entire length.

In one embodiment the cross-sectional diameter of the bottom circular base is about 10 mm or smaller, as such a dimension ensures that the anal sphincters are maintained contracted to such an extend that leakage are prevented during the administration procedure.

The ball-shaped insertion tip preferably has a diameter between about 0.8 cm and about 2.5 cm, preferably between about 1.0 and about 2.0 cm, and even more preferred between about 1.2 cm and about 1.5 cm, e.g. about 1.3 cm.

In a preferred embodiment of the enema nozzle according to the invention, the cross-sectional diameter of the ball shaped tip is about 13 mm, the cross-sectional diameter of the top base of the insertion body is about 4.5 mm, the cross-sectional diameter of bottom base is about 8 mm and the insertion body has a length of about 5 cm, as such dimensions will provide a convenient fit for most teenagers/adults.

Thus, the cross-sectional diameter of the enema nozzle according to the invention is relatively small compared to the cross-sectional diameter of the conventional enema nozzles, which often have a cross-sectional diameter of more than 1.5 cm. This ensures that the rectal ampulla and/or anal sphincters (including both the internal and external sphincter) of the user is not stretched, and accordingly remains tonically contracted whereby the enema nozzle according to the invention is securely retained in the rectum and that any fluids (liquid and gas) are maintained in the rectum for the desired period of time, and at least in the period it takes to deliver the enema, e.g. 3-5 minutes.

The insertion body may have any suitable length, depending on the intended delivery site of the enema in the rectum/colon, the longer the insertion body the further into the colon the enema may be delivered. It is however preferred that the insertion body have a length sufficient to place the ball-shaped insertion tip above the dentate line when placed in the rectum of a user, as this will prevent the ball-shaped insertion tip from irritating the sensitive nerve endings that may be positioned within the anal canal between the opening and the dentate line (also referred to as a pectinate line). Thus, is it preferred that the length of the insertion body (i.e. the elongated section of the enema nozzle intended for being placed inside the rectum), taken along the longitudinal axis of the elongated insertion body, is between 3 cm and 7 cm, preferably between 4 cm and 6 cm, and even more preferred about 5 cm as the inventors have found that such a length will place the ball-shaped insertion tip above the dentate line (for most users) and at the same time provide the unique self-retention of the enema nozzle by use of the anal sphincters.

Thus, using the above dimensions, the elongated insertion body will be relatively long and thin, and when the sphincters are closed around the elongated insertion body, any leakage of bodily fluids is not only prevented but the sphincters will also maintain the insertion body in the correct position in the rectum. Once the enema is administered the enema nozzle can be safely and quickly removed by a simply pulling action. The enema will together with stool etc. in the rectum build up pressure within the rectal ampulla (distend/stretch said rectal cavity), whereby the urge to eliminate the stool occurs. Thus, the user can use the restroom when the urge occurs and since the user neither has to deflate a retention member and/or worry about injuries when an expandable retention member folds in upon itself, the administration procedure, including the insertion and removal of the enema nozzle, is safe and can be performed quickly.

In order to deliver/expel the enema, the enema nozzle comprises at least one flow channel extending into at least one delivery opening, i.e. the at least one delivery opening is in flow communication with the at least one flow channel.

The flow channel of the enema nozzle may be in the form of one or more tube(s) placed inside the elongated insertion body and extending in the longitudinal direction of said insertion body, i.e. along the insertion body's longitudinal axis. However, in a preferred embodiment the at least one flow channel is defined by the side walls of the elongated insertion body, and if the flow channel extends into the ball-shaped insertion tip, also by the side walls of said ball-shaped insertion tip or a tubular opening in said insertion tip. This is particularly relevant when the ball-shaped insertion tip and the elongated insertion body are made as an integrated unit, as such an integrated unit can be formed as a hollow shell, where the inside space, or part of the inside space, forms the flow channel.

The one or more delivery opening(s) may in some embodiments be placed at the distal end of the insertion body, just below the top circular base, e.g. between 2 mm and 5 mm below said base. Thereby the ball-shaped insertion tip will function independently of the enema flow channel.

In some embodiments it may however be preferred to place said at least one delivery opening in the ball-shaped insertion tip, e.g. at the end of said tip. This may easily be obtained simply by extending the one or more interior flow channels into the ball-shaped insertion tip.

One preferred delivery opening, which can be used both in an enema nozzle according to the invention and in other enema nozzles, is a delivery opening in the form of a circumferical gap in the ball shaped insertion tip. Said circumferical gap is extending in a first plane perpendicular to the longitudinal axis of the insertion body. It is preferred that said first plane is placed closer to the distal end of the enema nozzle than a second plane passing through the center of the ball-shaped insertion tip, and wherein the first and second plane are parallel to each other. Such a circumferical gap allows the enema to be expelled into the body opening in all directions, which may be particularly desirable where the user's rectum or colon is inflamed or otherwise sensitive to contact by a pressurized stream of enema.

The circumferical gap may in one embodiment be divided into a number of openings, e.g. two, three or more openings along the periphery of the insertion body.

However, in a preferred embodiment the circumferical gap is a 360 degree circumferical gap, extending continuously along the entire circumference of the ball-shaped insertion tip along the first plane. In said embodiment the ball-shaped insertion tip comprises a first sphere part and a second sphere part, and wherein the first and second sphere parts are arranged for being connected to each other in such a way that the ball-shaped £ insertion tip is provided with a 360 degree circumferical gap between the two sphere parts

The first sphere part is: 7 formed as part of a sphere, e.g. shaped as a hollow sphere with an upper section, in the form of a sphere cap having a first height, missing. In this way a first opening is provided into the inside of the first sphere part defined by the walls of said first sphere part. The first sphere part is connected to the elongated insertion body at the top circular base, and the flow channel is extending into the inside of the first sphere part.

The second sphere part comprises a second sphere cap having a second height, and wherein said second height is smaller than the first height of the first sphere cap that was missing from the first sphere part, such that when the first and second sphere parts a combined into a substantially spherical shape, a circumferical gap is provided between said two sphere parts.

It is preferred that the outer surface of the first sphere part and the outer surface of the second sphere cap have the same radius of curvature such that a substantially ball-shaped insertion tip is provided when the two sphere parts are combined. It is further preferred that the circumferical gap along the outer edges, i.e. the edges facing the exterior surface of the ball-shaped enema nozzle is rounded such the provided ball-shaped insertion tip does not have any sharp edges or parts.

The two sphere parts can be connected to each other via any suitable connection system, the only requirement being that a delivery opening in the form of a 360 degrees circumferical gap between the two sphere parts is provided.

In a preferred embodiment the first sphere part comprises an internal space defined by the internal walls of the first sphere part, i.e. the internal space is in the form of a part of a sphere, and the second sphere part comprises a male coupling part arranged for being connected/attached to the internal walls of the first sphere part.

The male coupling part may in this respect comprise a protrusion comprising a plurality of legs e.g. three to eight legs each arranged for engaging the internal walls of the first sphere part. The space between the legs will provide openings between the internal walls of the first sphere part and the male coupling part allowing the enema to flow from the flow channel and out through the circumferical gap.

Each leg is preferably equidistantly spaced around the longitudinal axis of the male coupling part, which preferably is overlapping with the longitudinal axis of the enema nozzle. Each leg may be a plate like construction having a first edge which is complementary to part of the internal walls of the first sphere part, allowing the first and second sphere part to be firmly attached to each other, e.g. by gluing or ultrasonic welding. The inventors of the present invention have furthermore found that if each leg comprises an indention with a curvature extending towards the circumferical gap, the liquid administered via the circumferical gap will be administered via a swirling motion.

As an alternative to e.g. glue the first and second sphere parts together, the first sphere part may comprise a female coupling part, e.g. one or more barbs arranged on the internal walls of the first sphere, and the male coupling part may comprise one or more engagement means, e.g. teethe and/or hooks adapted to engage the one or more barbs in the female coupling part, such that when the male coupling part is inserted into the first sphere part, a snap fit between the male and female coupling parts are provided.

Irrespectively of how the first and second sphere parts are connected, it is preferred that they cannot be disengaged in any way after they are combined/connected.

The inventors of the present invention has found that an especially advantageously embodiment of the enema e nozzle according to the invention is provided when the circumferical gap is offset with an angle a of about 30-45 degrees from the longitudinal axis of the enema nozzle, preferable about 40 degrees, and when said circumferical gap is about 0.5-2 mm wide, preferably around 1 mm wide, taken along the outer surface of the ball-shaped insertion tip in the longitudinal direction of the enema nozzle, i.e. from the distal end towards the proximal end of the enema nozzle.

The enema nozzle is preferably arranged for being connected to a delivery container accommodating a liquid/enema, such that the delivery container communicates £ with the delivery opening(s) in order to administer the liquid, e.g. an enema to a patient/user. Said delivery container may be any suitable irrigation reservoir, e.g. an enema delivery container in a large bed-side irrigation system for use, in medical or hospital facilities and/or a small compact delivery container for home-administration of enema. The nozzle may be removable connected to the delivery container by means of any conventional coupling unit. Said coupling unit can e.g. comprise a first coupling part attach to the enema nozzle, e.g. via a tubular extension part in order to provide a distance between the elongated insertion body and the coupling unit, and a second, complementary, coupling part attached to the delivery container for providing a fluid communication between the two parts. In an especially simple embodiment the first coupling part is the tube for the flow channel and the second coupling part is a second tube on the delivery container. Said second tube is preferably adapted for providing a liquid tight fit together with the first tube in order to provide the coupling, e.g. obtained by a first and second tube made of a flexible material, such that one tube can be placed in the other tube.

In some embodiments it may be preferred to have the ball-shaped insertion tip made of one material, and the elongated insertion body of a different material. For instance the insertion body can be made of a relatively stiff material, thereby providing the longitudinal rigidity and strength required for easily advancing the nozzle in the body opening, and the ball-shaped insertion tip can be made of a softer, more elastic, and more flexible material such that the insertion tip can deform and/or flex slightly when it is inserted into the rectum and colon. However in an especially simple embodiment according to the invention, the ball-shaped insertion tip and the elongated insertion body are made of the same material, preferably as an integrated unit.

In order to provide the desired combination of rigidity/flexibility of the enema nozzle, said ball-shaped insertion tip and/or the insertion body, or at least a part of said insertion body, may be made of a polymer. Suitable materials in this respect are elastomeric polymers, preferably selected from the group consisting of thermoplastic elastomers (TPE), polypropylene (PP), polyurethane (PU), polyethylene (PE), polyvinyl chloride (PVC), silicone, styrene ethylene butylene styrene (SEBS), thermoplastic polyester elastomer (TPC), thermoplastic styrenic elastomer (TPS), and other similar polymers. The flexibility of the ball-shaped insertion tip and the insertion body respectively, can accordingly be altered, e.g. by changing the length of the polymer chains, creating branched chains from linear polymer chains, cross linking the polymer chains and/or adding plasticizers into the polymer.

The enema nozzle according to the present invention may be manufactured using any conventional process. It is however preferred that the enema nozzle is manufactured in a single injection molding procedure by 1K injection molding, 2K injection molding or 3K injection molding. 2K or 3K injection molding consists of processing two or three different polymers (or polymer compositions) into an end product by means of one injection molding process. With the 2K and 3K technique a possible assembly step of e.g. the ball-shaped insertion tip and elongated insertion body is prevented.

This technology enables a simple and effective manner of integrating the properties of the polymers into the enema nozzle according to the invention. For instance, the 2K injection molding process can be used to combine two different elastomeric polymers with different properties, e.g. one first elastomeric polymer that will form the insertion body and one second elastomeric polymer that will form the ball-shaped insertion tip.

The 2K or 3K injection molding process is preferably performed on one machine that is programmed to perform the injection of the polymers in a single cycle. The injection molding may either be a co-injection molding process, a sequential injection molding process or an over molding injection process.

The inventors of the present invention has found that inserting the enema nozzle according to the present invention into a body opening e.g. the rectum, requires significantly less force than inserting a conventional enema nozzle as the enema nozzle according to the invention is “sucked” into the rectum, and the present enema nozzle is therefore especially advantageously for disabled patients and/or patients suffering form an impairment e.g. relating to aging, diagnose or trauma. Such patients normally have reduced hand strength and/or dexterity, and the force needed to insert a conventional enema nozzle may have the consequence that the user is no longer able to use the device him-or herself. This is remedied by the present enema nozzle, which therefore is excellently suited for use at home. It is noted that in situations where the users normal anal sphincter function is compromised the enema nozzle may of cause be used, but the self-sealing, self-retaining effect surprisingly obtained with the enema nozzle according to the invention will not be observed to the same degree. This will O course depend of the degree of damage to the anal sphincter(s), and/or if other conditions is present at the insertion site, e.g. obstructions such as fistulas or abscesses.

It is preferred that the enema nozzle according to the invention does not comprise parts or elements that are arranged for chancing form and/or size during use. In this way the enema nozzle does not comprise any expandable parts, e.g. inflatable balloons and/or other retention members which may either fold out or expand during use, e.g. after insertion. Furthermore, since the retention member does not have to be inflated/deflated the time-consuming and complicated process of inflating and deflating the balloon is removed, thereby providing a fast and reliable method of using the enema nozzle according to the invention and ensuring that said nozzle is securely held in place during the entire administration procedure. Furthermore, the user does not have to worry about adjusting the amount of fluid in the retention member in order to provide the required expansion for retaining the nozzle in the rectum, and the enema nozzle will appear small and harmless especially in comparison with the more complicated enema systems having tubes for inflating/deflating a balloon. The risk of an inflatable balloon rupturing and thereby damaging the colon wall, is also eliminated.

In some embodiments, at least part of the enema nozzle intended for being inserted into the body cavity may be coated with a hydrophilic coating, for example either as a full coverage coating or an island coating consisting of hydrophilic dots separated by not-coated areas. When the hydrophilic coating gets wet, e.g. simply when contacted with water, saline or other liquid swelling medium prior to inserting the enema nozzle, the hydrophilic coating gels and/or swells and confers a friction-reducing surface to the exterior face of the coated section or coated part of the enema nozzle, thereby aiding in enabling a gentle insertion of the nozzle into e.g. the rectum. It is however preferred that it is only the ball-shaped insertion tip which comprises the hydrophilic coating in order to ensure that the self-retaining self-sealing effect of the enema nozzle according to the invention is not compromised.

The enema nozzle may comprise other features, e.g. a number of one-way valves for preventing back flow into the nozzle etc.

Even though the enema nozzle and enema system according to the invention is intended for administering an enema to the colon via the rectum, the enema nozzle and device may also be used for irrigation, cleansing and/or infusion into other body cavity, e.g. artificial stomas and fistulas.

The invention will be explained in greater detail below, describing only exemplary embodiments of the enema nozzle with reference to the drawing, in which

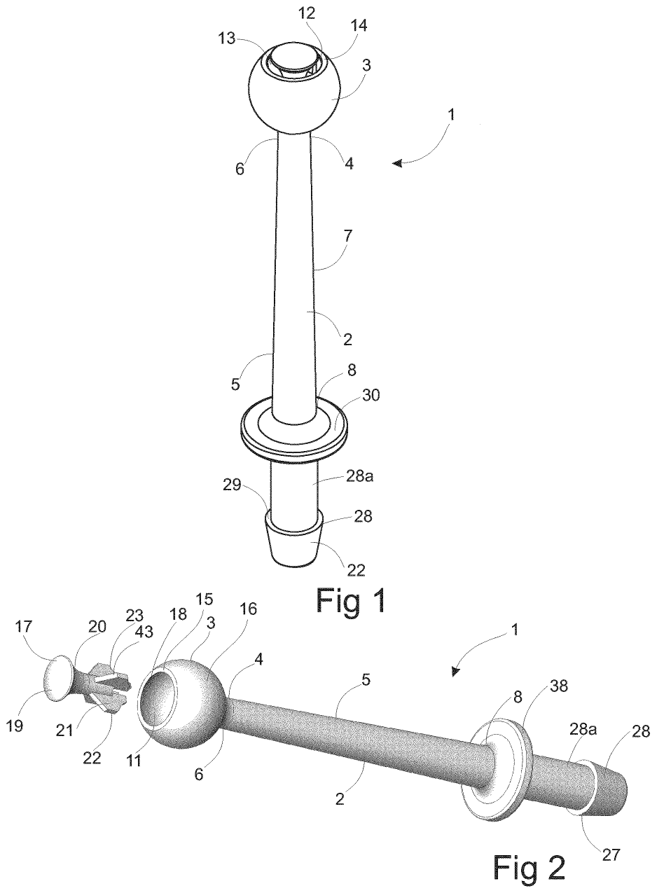

FIGS. 1-5 shows a first embodiment of an enema nozzle according to the present invention,

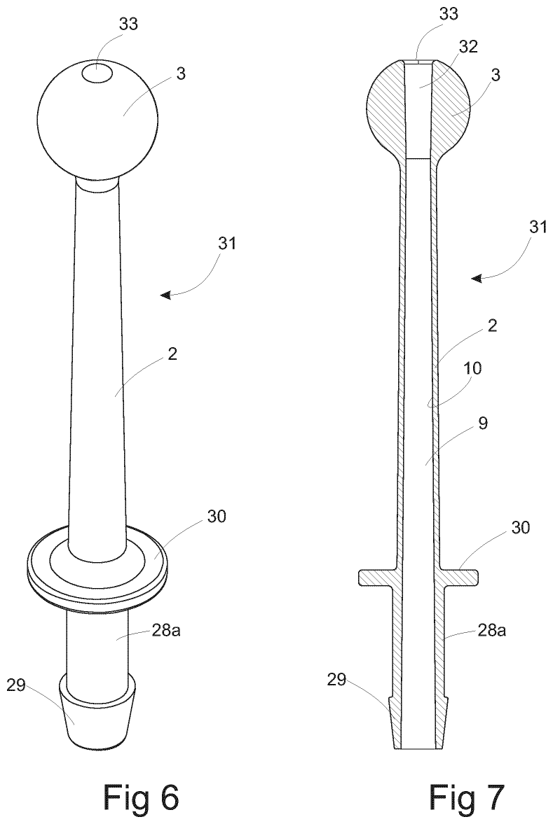

FIGS. 6 and 7 shows a second embodiment of an enema nozzle according to the present invention,

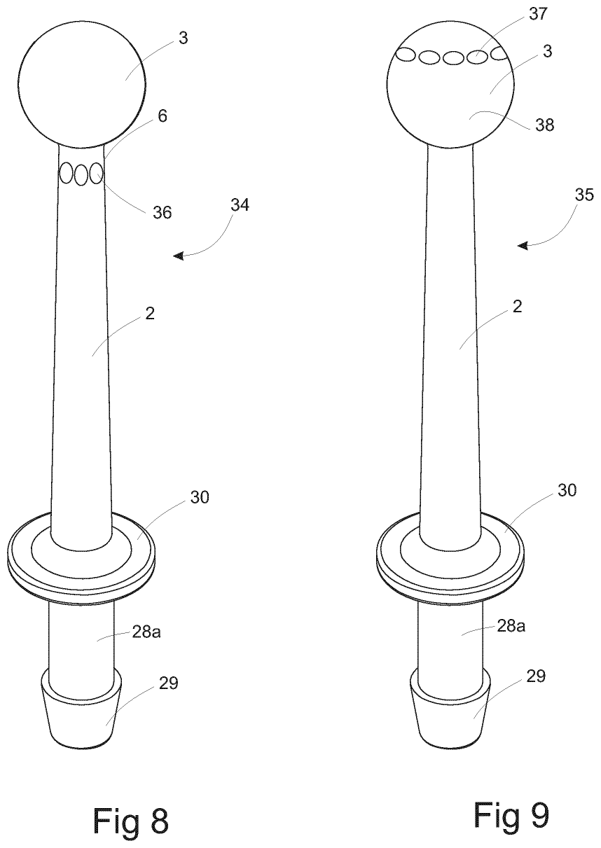

FIG. 8 shows a third embodiment of an enema nozzle according to the present invention,

FIG. 9 shows a fourth embodiment of an enema nozzle according to the present invention,

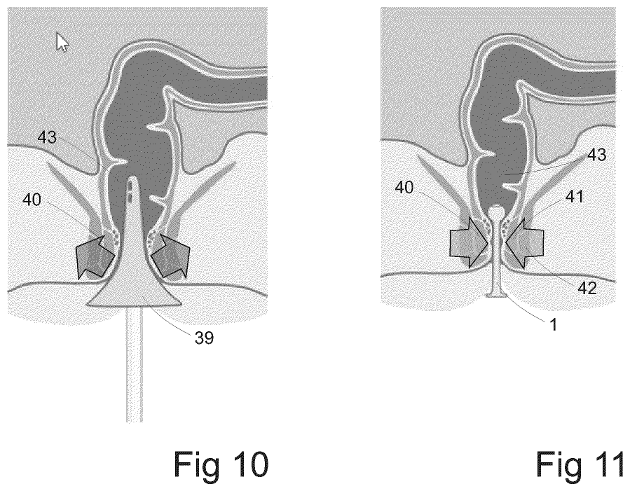

FIG. 10 shows a conventional enema nozzle inserted into the rectum/colon of a user, and

FIG. 11 shows an enema nozzle according to the present invention inserted into the rectum/colon of a user.

The invention is described below with the assumption that the entire enema nozzle is made of the same material, e.g. a polypropylene. This assumption is not to be construed as limiting, as the ball-shaped insertion tip and elongated insertion body in the embodiments described below also could be made as separate units/parts, e.g. of different materials, and wherein the separate units/parts are connected to each other by conventional means, e.g. welding, heat etc.

FIGS. 1-5 show a first embodiment of the enema nozzle 1 according to the invention. Said nozzle consists basically of an elongated insertion body 2 and a ball-shaped insertion tip 3 arranged at the distal end 4 of the insertion body 2.

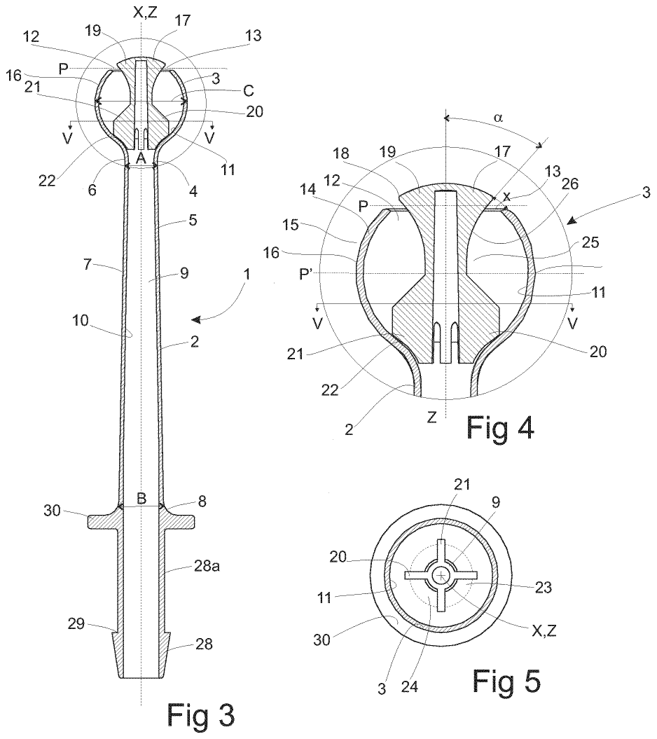

As best seen in FIG. 3, which is an cross-sectional view of the enema nozzle 1, the elongated insertion body 2 have the shape of a truncated cone 5, the top circular base 6 of the elongated insertion body (truncated cone 5) is connected/attached to the ball-shaped insertion tip 3 such that the side face 7 of the insertion body 2 extended outwards starting from the top circular base 6 towards the bottom circular base 8. The top circular base 6 has a smaller cross-sectional diameter A than the cross-sectional diameter B of the bottom circular base 8.

It is further evident from the figures, e.g. FIG. 3, that the cross-sectional diameters A, B of the insertion body 2 is smaller than the cross-sectional diameter C of the ball-shaped insertion tip 3, irrespectively of where said cross-sectional diameter A, B is taken/measured between the top and bottom base. The cross-sectional diameters A, B, C are all taken in a plane perpendicular to the longitudinal axis X of the enema nozzle 1.

The enema nozzle 1 comprises a flow channel 9, best seen in FIG. 3. Said flow channel 9 is defined by the internal side Walls 10 of the elongated insertion body 2 and the internal side walls 11 of the ball-shaped insertion tip 3. In the embodiment shown in FIG. 1-5 the delivery opening 12 is in the form of a 360 degree circumferical gap 13 continuously extending along the entire circumference 14 in an upper half 15 of the ball-shaped insertion tip 3 along a plane P perpendicular to the longitudinal axis X of the enema nozzle, such that the circumferical gap provides a 360 degree opening/gap 13 in the ball-shaped insertion tip 3, above a second plane P′ that coincides with a plane going through the center of the ball-shaped insertion tip 3.

In the first embodiment 1 of the enema nozzle according to the invention, the ball-shaped insertion tip 3 comprises a first sphere part 16 and a second sphere part 17.

The first sphere part 16 is connected to the elongated insertion body 2, and may either be integrated with the insertion body or attached to said insertion body by other means e.g. by gluing. Said first sphere part 16 have the shape of a hollow sphere with an upper section (in the form of a sphere cap having a first height) missing. In this way a first opening 18 is provided into the inside of f the first sphere part defined by the walls 11 of the first sphere part. The inside of the insertion body 2 defining the flow channel 9, is in flow communication with the inside of the first sphere part The second sphere part 17 comprises a second sphere cap 19 and a male coupling part 20 arranged for being inserted into the first opening 18 and being connected/attached to the internal walls 11 of the first sphere part 16.

The second sphere cap 17 has a second height which is smaller than the first height of the first sphere cap (missing from the first sphere part), such that when the first and second sphere parts 16,17 are connected to each other, a substantially spherical form is provided with the circumferical gap 13 between the two sphere parts 16,17.

The outer surface of the first sphere parts 16 have the same radius of curvature as the outer surface of the second sphere cap 19 such that a substantially ball-shaped insertion tip 3 is provided when the two sphere parts 16,17 are combined, with the circumferical gap 13 provided between said two sphere parts 16,17.

As best seen in FIGS. 2 and 5, the male coupling part 20 comprise four plate-like legs 21 equidistantly spaced around the longitudinal axis Z of male coupling part (which is coinciding with the longitudinal axis X of the enema nozzle) and each having a first edge 22 which is complementary to part of the internal walls 11 of the first sphere part 16, allowing the first and second sphere part to be firmly attached to each other, e.g. by gluing or ultrasonic welding.

The space 23 provided between the legs 21 of the male coupling part 20 and the internal walls 11 of the first sphere part 16, will provide four flow openings 24 allowing the enema to flow from the flow channel 9 and out through the circumferical gap 13. These flow openings 24 are best shown in FIG. 5 which a cross-section taken along the lines V-V in FIGS. 3 and 4. FIG. 4 is an enlarged view of the ball-shaped insertion tip shown in FIG. 3.

As shown in FIG. 4, each leg has an indention 25 with a curvature 26 extending towards the circumferical gap 13, which allows the liquid administered via the circumferical gap 13 to be set out in a swirling motion.

The circumferical gap 13 is preferably offset with an angle of α, e.g. 30-45 degrees, from the longitudinal axis of the enema nozzle, preferable about 40 degrees. The circumferical gap preferably has a width x, e.g. about 0.5-2 mm, e.g. 1 mm, taken along the outer surface 27 of the ball-shaped insertion tip.

The enema nozzle 1 is at the proximal end of the enema nozzle 2 arranged for being removable connected to any suitable irrigation or enema delivery container by means of coupling unit 28 which is connected to/integrated with the elongated insertion body via a tubular extension part 28a in order to provide a suitable distance between the elongated insertion body and the coupling unit 28. Said coupling unit comprises a first coupling part 29 attach to the enema nozzle 1 and a second coupling part on the enema delivery container (not shown) that is complementary to the first coupling part 29.

In order to prevent the enema nozzle 1 from being placed/sucked to far into the rectum the enema nozzle 1 may comprises a flared stop plate 30, placed in the intersection between the bottom circular base 8 of the elongated insertion body 2 and the tubular extension part 28a such that the ball-shaped insertion tip 3 after insertion will be placed above the dentate line of a user.

FIGS. 6 and 7 show a second embodiment 31 of the enema nozzle according to the invention. Said embodiment corresponds mainly to the first embodiment 1 and for like parts the same reference numbers are used.

In the second embodiment 31 the ball-shaped insertion tip 3 and the elongated insertion body 2 are made as an integrated unit wherein the flow channel 9 is defined by the internal side walls 10 of the elongated insertion body 2 and a tubular channel 32 extending though the ball-shaped insertion tip 3 and ends in a central delivery opening 33 placed at the distal end of the ball shaped insertion tip 3.

FIGS. 8 and 9 show a third embodiment 34 and a fourth embodiment 35 respectively of the enema nozzle according to the invention. Said embodiments 34, 35 corresponds basically to the embodiment 31 in FIGS. 6 and 7, and for like parts the same reference numbers are used. The main differences between the second, and the third and fourth embodiments, is that the third only have one delivery opening whereas the third and fourth embodiment have several delivery openings.

In the third embodiment 34 shown in FIG. 8 the flow channel 9 is also defined by the internal walls 10 of the insertion body, as for the second embodiment, but ends in a number of delivery openings 36 placed at the distal end of the insertion body 2, i.e. just below the top circular base 6, e.g. between 2 mm and 5 mm below said base. Thus, the flow channel 9 does not extend into the ball-shaped insertion tip 3 in this embodiment.

In the fourth embodiment 35, shown in FIG. 9 the flow channel 9 extends into the ball-shaped insertion tip 3 but is arranged such that it ends in a number of delivery openings 37 placed in the side wall 38 of the ball-shaped insertion tip 3.

As is evident from FIGS. 1-9 the enema nozzle 1; 31; 34; 35 according to the invention is relatively long and thin enabling said enema nozzle to be retained in the rectum of a user simply using the normal function of the user's anal sphincters.

The difference between the use of a conventional enema nozzle e.g. in the form of a trumpet head, and the enema nozzle 1; 31; 34; 35 according to the invention is illustrated in FIGS. 10 and 11.

As illustrated by arrows in FIG. 10 a conventional enema nozzle 39 will stretch the anal sphincter muscles 40 to such a degree that the anal sphincters cannot contracted sufficiently to maintain the enema nozzle 40 in place and/or the fluids in the rectum.

In contrast, the unique combination of a thin elongated insertion body 2 with the shape of a slightly truncated cone and a larger ball-shaped insertion tip 3, as for the enema nozzle 1; 31; 34; 35 according to the invention, ensures that the anal sphincters (both internal 41 and external 42), which are closed in the natural, resting state, naturally draws the ball-shaped insertion tip in, and then close behind it. This is illustrated by the arrows in FIG. 11.

The user's anal sphincters 4 will accordingly remain contracted and hold themselves in the substantially contracted condition thereby not only keeping the enema nozzle 1; 31; 34; 35 in place, but also ensure that the irrigation liquid and bodily excretions are maintained in the rectum/colon 43 during the enema administration procedure.

The inventors of the present invention have also found that the insertion of the enema nozzle 1; 31; 34; 35 into the anus/rectum of the patient is even easer than with the known enema nozzles 39 in the art. The unique shape of a ball-shaped insertion tip 3 and a elongated truncated insertion body 2 ensures that during insertion the squeezing action of the anal sphincter 40 against the insertion body 2 pushes (sucks) the enema nozzle 1; 31; 34; 35 into the rectum 43, thereby ensuring that the enema nozzle according to the invention will be placed in the correct position in the rectum/colon 43.

The person skilled in the art will understand that the dimensions of the embodiments shown in the figures may vary, but in one preferred embodiment of the enema nozzle 1 shown in FIGS. 1-5 , the ball-shaped insertion tip 3 has a cross-sectional diameter of about 13 mm, the elongated truncated insertion body 2 a length of about 5 cm, the cross-sectional diameter of the top circular base 6 is about 4.5 mm, i.e. about 2.6 times smaller than the cross-sectional diameter of the ball-shaped insertion tip 3, and the cross-sectional diameter of the bottom circular base 8 is about 8 mm, i.e. about 1.6 times smaller than the cross-sectional diameter of the ball-shaped insertion tip 3. The circumferical gap 13 is offset with 40 degrees from the longitudinal axis X of the enema nozzle, and said gap is about 1 mm, taken along the outer surface 27 of the ball-shaped insertion tip 3.

The enema nozzles disclosed herein may be used in small hand held enema devices, e.g. connected to a hand held delivery pump, or used for larger irrigation systems wherein the enema nozzle is connected to the enema reservoir via a relatively long delivery tube, and wherein the irrigation system e.g. comprises pumping means, control means, collection means for the stool, etc. The enema nozzle may be a disposable nozzle or the nozzle can be individually cleaned and/or sterilised.

Modifications and combinations of the above principles and designs are foreseen within the scope of the present invention.

What is claimed is:

1-21. (canceled)

22. An enema nozzle arranged for delivering an enema to a user, said enema nozzle comprises a ball-shaped insertion tip and an elongated insertion body having the shape of a truncated cone, and wherein the cross-sectional diameter of the insertion body is smaller than the cross-sectional diameter of the ball-shaped insertion tip, wherein said ball-shaped insertion tip is connected to a top circular base of the insertion body and the side face of the insertion body extends outwards starting from the top circular base towards a bottom circular base and wherein the ball-shaped insertion tip has a cross-sectional diameter that is about 2 to 5 times larger than the cross-sectional diameter of the top circular base of the insertion body, and wherein the enema nozzle does not comprise parts or elements that are arranged for chancing form and/or size during use, such as expandable parts and/or inflatable balloons.

23. An enema nozzle according to claim 22, wherein the cross-sectional diameter of the insertion body is smaller than the cross-sectional diameter of the ball-shaped insertion tip in the entire length of the insertion body.

24. An enema nozzle according to claim 22, wherein the ball-shaped insertion tip has a cross-sectional diameter that is about 2.6 times the cross-sectional diameter of the top circular base of the insertion body.

25. An enema nozzle according to claim 22, wherein the bottom circular base have a cross-sectional diameter which is about 1.1 to 3.5 times larger than the cross-sectional diameter of the top circular base, preferable about 1.2 to 2 times, even more preferred about 1.6 times larger.

26. An enema nozzle according to claim 22, wherein the cross-sectional diameter of the bottom circular base is 10 mm or smaller.

27. An enema nozzle according to claim 22, wherein the ball-shaped insertion tip has a cross-sectional diameter between 0.8 cm and 2.5 cm, preferably between 1.0 and 2.0 cm, and even more preferred between 1.2 cm and 1.5 cm, e.g. 1.3 cm.

28. An enema nozzle according to claim 22, wherein the cross-sectional diameter of the ball-shaped insertion tip is about 13 mm, the cross-sectional diameter of the top base of the insertion body is about 4.5 mm, the cross-sectional diameter of bottom base is about 8 mm and the insertion body has a length of about 5 cm.

29. An enema nozzle according to claim 22, wherein the enema nozzle comprises at least one flow channel and at least one delivery opening in flow communication with the at least one flow channel.

30. An enema nozzle according to claim 29, wherein the flow channel is defined by the side walls of the elongated insertion body, and by the side walls of the ball-shaped insertion tip or by a tubular channel in said insertion tip.

31. An enema nozzle according to claim 29, wherein the at least one delivery opening is formed as a circumferical gap in the ball-shaped insertion tip and wherein said circumferical gap is extending in a first plane perpendicular to the longitudinal axis (X) of the insertion body.

32. An enema nozzle according to claim 31, wherein said first plane is placed closer to the distal end of the enema nozzle than a second plane passing through the center of the ball-shaped insertion tip, and wherein the first and second planes are parallel to each other.

33. An enema nozzle according to claim 31, wherein the ball-shaped insertion tip comprises a first sphere part and a second sphere part comprising a second sphere cap, and wherein the first and second sphere parts are arranged for being connected to each other in such a way that the ball-shaped insertion tip is provided with a 360 degree circumferical gap provided between the two sphere parts.

34. An enema nozzle according to claim 33, wherein the outer surface of the first sphere parts and the outer surface of the second sphere cap have a same radius of curvature.

35. An enema nozzle according to claim 33, wherein the first sphere part comprises an internal space defined by the internal walls of the first sphere part, and the second sphere part comprises a male coupling part arranged for being connected/attached to the internal walls of the first sphere part.

36. An enema nozzle according to claim 31, wherein the circumferical gap is offset with an angle a of about 30-45 degrees from the longitudinal axis of the enema nozzle, preferable about 40 degrees, and wherein the circumferical gap is about 0.5-2 mm wide, preferably around 1 mm wide, taken along the outer surface of the ball-shaped insertion tip in the longitudinal direction of the enema nozzle.

37. An enema nozzle according to claim 22, wherein the enema nozzle is arranged for being connected to a delivery container accommodating a liquid.

38. An enema system comprising the enema nozzle according to claim 22.

39. A method of manufacturing the enema nozzle according to claim 22, wherein said enema nozzle is manufactured by 1K injection molding, 2K injection molding or 3K injection molding.

40. A method of assembling the enema nozzle according to any of the claims 35, wherein the first sphere part comprises an internal space defined by the internal walls of the first sphere part, and the male coupling part of the second sphere part comprises a number of plate-like legs, wherein said method comprises placing the male coupling part inside the internal space of the first sphere part and attaching a first edge of each leg to a complementary section on the internal walls of the first sphere part.
